(19) United States
(12) Patent Application Publication
(10) Pub. No.: US 2002/0188275 A1
(43) Pub. Date: Dec. 12, 2002

(54) Multi Directional infusion needle

(76) Inventor: James F. McGuckin JR., Radnor, PA (US)

Correspondence Address: Neil D. Gershon, Chief Patent Counsel, Rex Medical, 2023 Summer St. Suite 2, Stamford, CT, 06905, US

(21) Appl. No.: 09/800,332

(22) Filed: Mar. 5, 2001

Related U.S. Application Data
(63) Continuation of application No. 09/631,778, filed on Aug. 3, 2000.
(63) Continuation of application No. 09/668,067, filed on Sep. 22, 2000, now Pat. No. 6,425,887.
Application No. 09/668,067 is a division of application No. 09/457,844, filed on Dec. 9, 1999.
(60) Provisional application No. 60/111,624, filed on Dec. 9, 1998.
(60) Provisional application No. 60/130,597, filed on Apr. 22, 1999.

(51) Int. Cl. A61M 5/178

(52) U.S. Cl. 604/506; 604/164.01

(57) ABSTRACT

A method of treating a tumor in a patient's liver or kidney comprising providing an introducer, providing an elongated member having a plurality of needles extending therefrom, inserting the introducer into the patient to a position adjacent the tumor, advancing the elongated member with respect to the introducer so the plurality of needles extend from a distal end of the introducer into the tumor, and injecting ethanol through a lumen in each of the needles so the ethanol exits through an opening in the needle and infuses into the tumor to ablate the tumor.

### BACKGROUND AND SUMMARY OF THE INVENTION

Tumor ablation involves non-surgical treatment of inoperable tumors or lesions and includes treatment options such as cryotherapy and high energy ultrasound ablation. Another ablation method used for carcinoma of the liver and kidney is percutaneous ethanol injection. Originally introduced as a palliative treatment for inoperable hepatocellular carcinoma of the liver, ethanol injection has now been shown to have curative potential comparable to resection in many patients, especially for smaller tumors. Current practice has been to directly inject ethanol into masses and allow the ethanol to infuse from one or more side holes into the tissue. The problem is that the infusion may not penetrate any deeper than the needle tract and thus, portions of the tumor are not effectively treated. The present invention solves this problem with a plurality of curved infusion needles made of a shape memory alloy such as nitinol, that can be deployed within an introducer cannula within the tumor where they radiate outward into an umbrella shape. Therefore, infusion can take place at multiple points within the tumor to provide wider dispersion of the ethanol. Following treatment, they are withdrawn into the cannula and the device is removed from the patient.

### BRIEF DESCRIPTION OF THE DRAWING

FIG. 1 depicts a perspective view of an embodiment of the present invention;

FIG. 2 depicts a side view of the multi-directional infusion needle of FIG. 1;

FIG. 3 depicts a side view of the needle of FIG. 2 being partially deployed;

FIG. 4 depicts a side view of the trocar introducer used with the embodiment of FIG. 1; and

FIG. 5 depicts a side view of the proximal assembly of FIG. 1;

### DETAILED DESCRIPTION

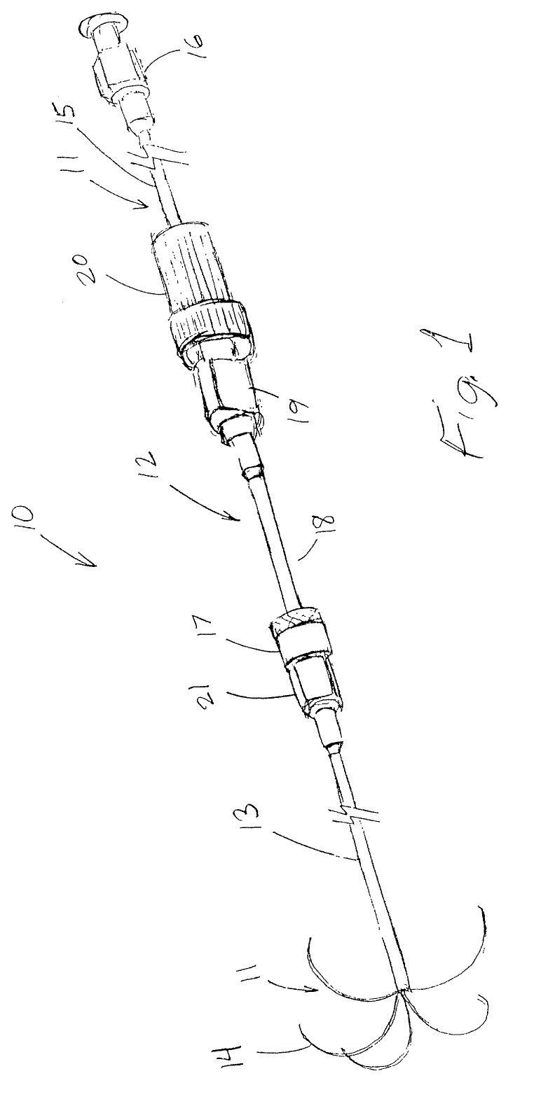

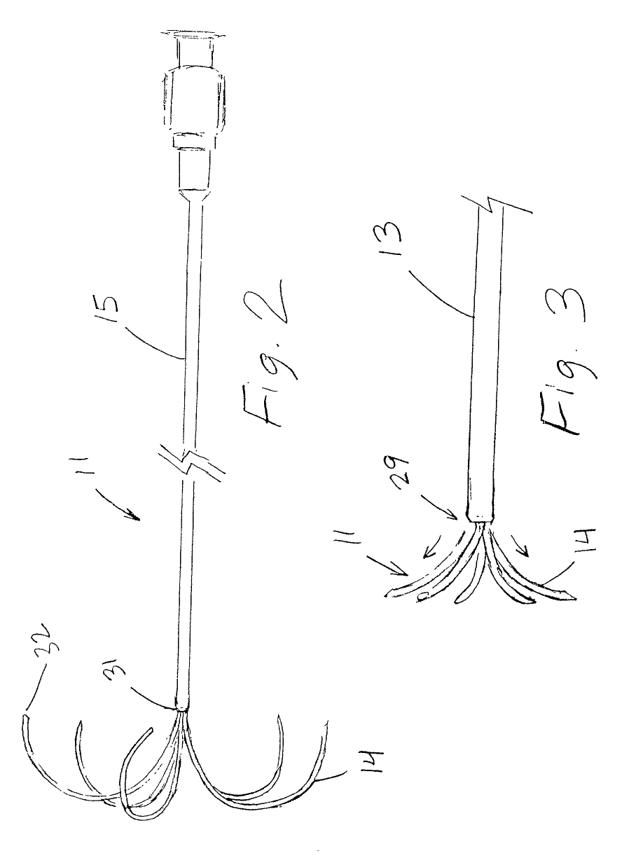

FIG. 1 depicts a perspective view of the multi-directional infusion needle assembly 10 of the present invention comprising a multi-directional infusion needle 11, a proximal assembly 12, and an introducer cannula 13. The multi-directional infusion needle 11, shown in FIG. 2, includes a needle cannula 15 affixed to a proximal hub 16 such as a standard female luer fitting. A plurality of hollow, curved infusion needles 14 are manifolded into the needle cannula, evenly spaced in an umbrella shape, and affixed in place with a solder joint 31. In the preferred embodiment, five curved infusion needles 14 are used. The curved infusion needles are preferably made of a shape memory alloy such as nitinol. The curved shape can be the result of cold working the nitinol by mechanically stressing the austenitic state material until it permanently deforms by producing a localized region of the martensitic form of nitinol. An alternative method is the standard heat forming method of shaping nitinol where the material is put under stress and subjected to high temperatures until the bend is maintained. While other metals such as stainless steel may be utilized, nitinol and other shape memory alloys allow the needles to retain their shape and more fully deploy outward when deployed. In the preferred embodiment, the needle cannula 15 has an O.D. of approximately 0.072″ and an I.D. of around 0.06″, while the individual curved infusion needles 14 have an O.D. of 0.02″ and I.D. of about 0.12″. The tips 32 of the curved infusion needles 14 are preferably beveled to better penetrate tissue.

Deployment of the curved infusion needles 14 of the multi-directional infusion needle 11 is depicted in FIG. 3. The curved infusion needles 14 are restrained by the introducer cannula 13 until the multi-directional infusion needle 11 is advanced, exposing the curved infusion needles 14 whereby they radiate outward to assume the umbrella configuration 33 of FIG. 2.

FIG. 4 depicts a side view of a introducer trocar assembly 23 used in placement of the multi-directional infusion needle assembly 10. It is comprised of the introducer cannula 13 of FIG. 1 and an inner stylet 22 having a proximal male hub 24 that fits into the proximal female hub 22 of the introducer cannula 13 when the inner stylet 22 is fully advanced into the lumen 28 of the introducer cannula 13. The inner stylet 22 includes a sharp tip 30 for penetrating tissue, preferably a diamond shape tip as depicted. The introducer trocar assembly 23 is introduced percutaneously into the liver or kidney and placed at the desired treatment location. The inner stylet 22 is then removed. The proximal assembly 12 with the preloaded multi-directional infusion needle 11 is then advanced into the introducer cannula 13 which remains in the patient. In the preferred embodiment, the introduced cannula has an O.D. of 0.095″ and I.D. of about) 0.076″, while the O.D. of the inner stylet is about 0.068″.

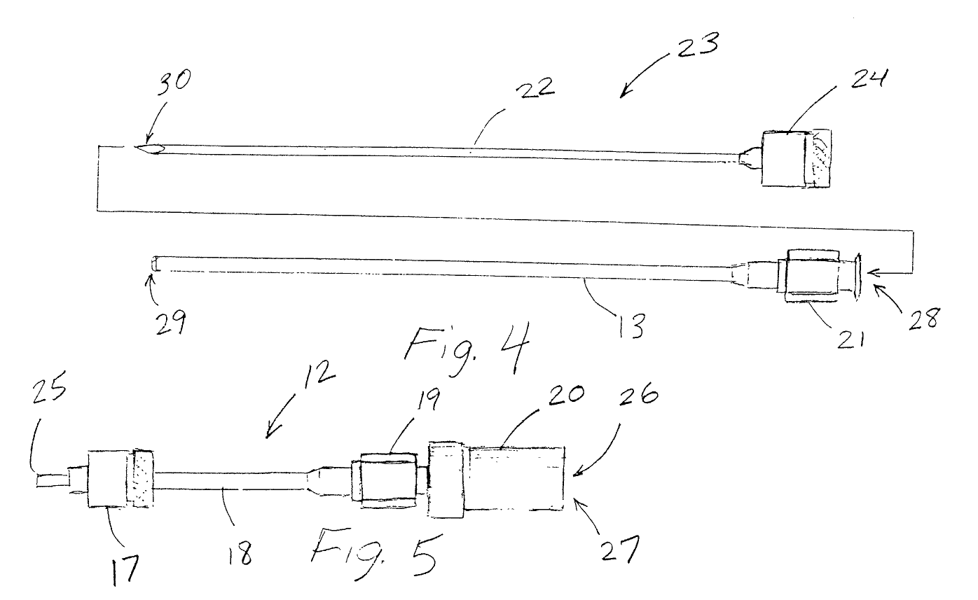

FIG. 5 depicts a side view of the proximal assembly of FIG. 1. The proximal assembly 12 includes a distal male adaptor 17 connected to an intermediate cannula 18 that is sufficiently large to accommodate the multi-directional infusion needle 11. At the proximal end of the intermediate cannula 18 is proximal assembly female adaptor 19 which is connected proximally to the proximal assembly hub 20, which in the illustrative embodiment is comprised of a Tuohy-Borst adaptor. The proximal assembly hub 20 is utilized by the physician during manipulation of the device. The multi-directional infusion needle 11 of FIG. 2 is loaded into lumen 26 at the proximal end 27 of the proximal assembly hub 20, the curved infusion needles 14 remaining within the intermediate cannula 18. The distal end 25 of the proximal assembly 12 with the preloaded multi-directional infusion needle 11 is then inserted into the proximal hub 21 of the introducer cannula as depicted in FIG. 1. The multi-directional infusion needle 11 is then advanced from the proximally assembly into the introducer cannula 13 where is deployed as depicted in FIGS. 1 and 3. Ethanol is infused into the multi-directional infusion needle assembly 10 via the proximal hub 16 of the multi-directional infusion needle 11. Following treatment, the multi-directional infusion needle 11 is withdrawn into the introducer cannula 13 and the entire assembly is removed from the patient.

What is claimed is:

1. A method of treating a tumor in a patient's liver or kidney comprising:
providing an introducer;
providing an elongated member having a plurality of needles extending therefrom, the elongated member dimensioned for receipt within the introducer;
inserting the introducer into the patient to a position adjacent the tumor;
advancing the elongated member with respect to the introducer so the plurality of needles extend from a distal end of the introducer into the tumor; and
injecting ethanol through a lumen in each of the needles so the ethanol exits through an opening in the needle and infuses into the tumor to ablate the tumor.

2. The method of claim 1, further comprising the step of withdrawing the extended needles into the introducer after infusion of the ethanol into the tumor.

3. The method of claim 1, wherein the introducer has a penetrating tip, and the step of inserting the introducer into the patient includes the step of percutaneously inserting the introducer to pierce tissue.

4. The method of claim 1, wherein the needles have a preformed bend so that the step of advancing the needles enable them to move to their preformed bent position.

5. The method of claim 4, wherein advancing the elongated member with respect to the introducer advances a plurality of needles radially outwardly with respect to the introducer.

6. The method of claim 5, wherein the needles have a preformed bend so that the step of advancing the needles enable them to move to their preformed bent position.

7. The method of claim 6, wherein the introducer has a penetrating tip, and the step of inserting the introducer into the patient includes the step of percutaneously inserting the introducer to pierce tissue.

8. The method of claim 7, further comprising the step of withdrawing the extended needles into the introducer after infusion of the ethanol into the tumor.

9. A method of treating a tumor in a patient's liver or kidney comprising:
providing an introducer;
providing an elongated member having a needle extending therefrom wherein the needle includes a lumen and an infusion opening, the elongated member dimensioned for receipt within the introducer;
inserting an introducer into the patient to a position adjacent the liver or kidney tumor;
advancing the elongated member with respect to the introducer so that the needle extends radially from the introducer into the tumor;
injecting ethanol through the lumen in the needle so the ethanol exits through the infusion opening in the needle and infuses into the liver or kidney tumor to ablate the tumor.

10. The method of claim 9, wherein the introducer has a penetrating tip and the step of inserting the introducer includes the step of percutaneously inserting the introducer through the patient's skin to penetrate tissue.

11. The method of claim 10, wherein the needle has a preformed bend so that the step of advancing the needle enables it to move to its preformed bent position.

12. The method of claim 11, wherein advancing the elongated member with respect to the introducer advances the needle radially outwardly with respect to the introducer.

13. The method of claim 12, further comprising the step of withdrawing the extended needle into the introducer after infusion of the ethanol into the liver or kidney tumor.
